(19) United States
(12) Patent Application Publication
(10) Pub. No.: US 2019/0006042 A1
(43) Pub. Date: Jan. 3, 2019

(54) A MEDICAL DATA MANAGEMENT METHOD, APPARATUS AND MEDICAL DATA SYSTEM

(71) Applicant: BOE TECHNOLOGY GROUP CO., LTD., Beijing (CN)

(72) Inventor: Lvwei WANG, Beijing (CN)

(21) Appl. No.: 15/744,746

(22) PCT Filed: Aug. 3, 2017
(86) PCT No.: PCT/CN2017/095823

§ 371 Date: Jan. 12, 2018

(30) Foreign Application Priority Data
Aug. 19, 2016 (CN) 201610696988.9

(51) Int. Cl. G16H 40/20; G16H 10/60; G16H 40/60

(52) U.S. Cl. CPC G16H 40/20; G16H 15/00; G16H 40/60; G16H 10/60

(57) ABSTRACT

Embodiments of the disclosure provide a medical data management method, apparatus and corresponding medical data system. The method comprises: a management mainframe creating a virtualized container of the medical data system, wherein the management mainframe is arranged with a user interface for managing the medical data system, the management mainframe configuring the virtualized container in N sub-nodes to form a distributed data system, and the management mainframe storing the medical data in a hospital information system HIS in the distributed data system, to facilitate a user to collect and enter corresponding medical data from the distributed data system via the user interface when conducting medical data analysis. The method may be applied in the medical data analysis process.

### RELATED APPLICATION

This application claims the priority of the Chinese patent application 201610696988.9 submitted to the China Patent Office on Aug. 19, 2016, of which the whole content is incorporated herein by reference.

### FIELD OF THE INVENTION

The disclosure relates to the field of medical technology, and in particular, to a medical data management method, apparatus and corresponding medical data system.

### BACKGROUND OF THE INVENTION

A HIS (hospital information system) refers to a computer application system for conducting information management and online operations in hospital management and medical activities. By establishing the HIS, major hospitals may collect and process clinical medical information of patients, enrich and accumulate clinical medical knowledge, and meanwhile, may also manage the administrative affairs of the hospitals, alleviate the labor intensity of the affair handling personnel, and assist the hospital in management.

For example, each time each patient seeks medical advice, the medical data produced in the whole process from registration to diagnosis and treatment will be recorded in the HIS of a hospital, and in the process of treatment, the doctor may determine a treatment solution in line with the actual physical condition of the patient in combination with the previous medical data.

However, when a doctor needs to conduct data analysis on the medical data, for example, analyze the relationship between diabetes and dietary habits, it is often necessary for the doctor to manually collect related medical data from the HIS and conduct medical analysis. Such a data analysis method is not only time-consuming and laborious, but may also cause problems of an inaccurate analysis result, etc. because the data collection is not comprehensive and so on.

Especially, with the sharp increase in the amount of medical data, the need for a doctor to conduct analysis on the medical data is more urgent. However, currently developed various big data analysis software and analysis methods require a user to master a high level of computer technology, and even require him to master much underlying code of the computer system to acquire and analyze the medical data, which results in that it is very difficult for the doctor to use it.

### SUMMARY OF THE INVENTION

Embodiments of the invention provide a medical data management method, apparatus and corresponding medical data system, which may simplify the data acquisition process at the time of medical data analysis, reduce the complexity of the medical data analysis, and improve the accuracy of the medical data analysis.

To achieve the above objectives, the embodiments of the invention adopt the following technical solutions.

In an aspect, an embodiment of the invention provides a management mainframe applied in a medical data system, which system comprises the management mainframe and N sub-nodes all connected with the management mainframe, wherein N is an integer greater than 0, the management mainframe comprising: a storage for storing data and an instruction, and a processor configured to, when the instruction is executed in the processor, implement the following steps of: creating a virtualized container of the medical data system, wherein the management mainframe is arranged with a user interface for managing the medical data system; configuring the virtualized container in the N sub-nodes to form a distributed data system; and storing the medical data in a hospital information system HIS in the distributed data system, to facilitate a user to operate the user interface to acquire the medical data required for conducting medical data analysis via the distributed data system.

Further, the virtualized container comprises a database for data storage and a data replication application for data replication, and at this point, the replication unit is used for sending a data replication instruction to at least one of the N sub-nodes, such that the sub-node receiving the data replication instruction stores first medical data into the database via the data replication application, wherein the first medical data is part of the medical data in the HIS.

Further, the processor is further configured to receive the database address information of the HIS inputted by the user on the user interface; and carry the database address information in the data replication instruction.

Further, the virtualized container comprises a data computation application for data computation and a data analysis application for data analysis, and at this point, the processor is further configured to receive a data analysis instruction triggered by the user on the user interface, in which data analysis instruction is comprised the feature information of the medical data analysis for this time; formulate M analysis tasks for accomplishing the medical data analysis for this time according to the data analysis instruction, wherein M is an integer greater than 0 and less than or equal to N; send the M analysis tasks to M sub-nodes of the N sub-nodes, such that the sub-nodes receiving the analysis tasks invoke the data computation application and the data analysis application to perform the received analysis tasks to obtain an analysis result; and display the analysis result on the user interface.

Further, the processor is further configured to acquire the address information of each of the N sub-nodes inputted by the user on the user interface; and configure the virtualized container in a corresponding sub-node according to the address information of each sub-node.

Further, the processor is further configured to, after configuring the virtualized container in the N sub-nodes to form a distributed data system, acquire an add-sub-node instruction, which carries the address information of a newly added sub-node; and configure the virtualized container in the newly added sub-node according to the address information of the newly added sub-node.

Further, the processor is further configured to, after configuring the virtualized container in the N sub-nodes to form a distributed data system, acquire a delete-sub-node instruction, which carries the address information of a to-be-deleted sub-node; and delete the virtualized container configured in the to-be-deleted sub-node according to the address information of the to-be-deleted sub-node.

Further, the virtualized container is a Docker container.

In a further aspect, an embodiment of the invention provides a medical data system comprising any of the management mainframes as described above and N sub-nodes all connected with the management mainframe, wherein N is an integer greater than 0.

In a still further aspect, an embodiment of the invention provides a medical data management method applied in a medical data system, which system comprises a management mainframe and N sub-nodes all connected with the management mainframe, wherein N is an integer greater than 0, the method comprising: the management mainframe creating a virtualized container of the medical data system, wherein the management mainframe is arranged with a user interface for managing the medical data system; the management mainframe configuring the virtualized container in the N sub-nodes to form a distributed data system; and the management mainframe storing the medical data in a HIS in the distributed data system, to facilitate a user to acquire the medical data required for conducting medical data analysis via the distributed data system when operating the user interface.

Further, the virtualized container comprises a database for data storage and a data replication application for data replication, wherein the management mainframe storing the medical data in the HIS in the distributed data system comprises: the management mainframe sending a data replication instruction to at least one of the N sub-nodes, such that the sub-node receiving the data replication instruction stores first medical data into the database via the data replication application, wherein the first medical data is part of the medical data in the HIS.

Further, before the management mainframe sending a data replication instruction to at least one of the N sub-nodes, there is further comprised: the management mainframe receiving the database address information of the HIS inputted by the user on the user interface; and the management mainframe carrying the database address information in the data replication instruction.

Further, the virtualized container comprises a data computation application for data computation and a data analysis application for data analysis, wherein after the management mainframe storing the medical data in the HIS in the distributed data system, there is further comprised: the management mainframe receiving a data analysis instruction triggered by the user on the user interface, in which data analysis instruction is comprised the feature information of the medical data analysis for this time; the management mainframe formulating M analysis tasks for accomplishing the medical data analysis for this time according to the data analysis instruction, wherein M is an integer greater than 0 and less than or equal to N; the management mainframe sending the M analysis tasks to M sub-nodes of the N sub-nodes, such that the sub-nodes receiving the analysis tasks invoke the data computation application and the data analysis application to perform the received analysis tasks to obtain an analysis result; and the management mainframe displaying the analysis result on the user interface.

Further, the management mainframe configuring the virtualized container in the N sub-nodes comprises: the management mainframe acquiring the address information of each of the N sub-nodes inputted by the user on the user interface; and the management mainframe configuring the virtualized container in a corresponding sub-node according to the address information of each sub-node.

Further, after the management mainframe configuring the virtualized container in the N sub-nodes to form a distributed data system, the method further comprises: the management mainframe acquiring an add-sub-node instruction, which carries the address information of a newly added sub-node; and the management mainframe configuring the virtualized container in the newly added sub-node according to the address information of the newly added sub-node.

Further, after the management mainframe configuring the virtualized container in the N sub-nodes to form a distributed data system, there is further comprised: the management mainframe acquiring a delete-sub-node instruction, which carries the address information of a to-be-deleted sub-node; and the management mainframe deleting the virtualized container configured in the to-be-deleted sub-node according to the address information of the to-be-deleted sub-node.

Further, the virtualized container is a Docker container.

In a yet still further aspect, an embodiment according to the invention provides a management system applied in a medical data system, which system comprises the management system and N sub-nodes all connected with the management system, wherein N is an integer greater than 0, the management system comprising: a creation unit for creating a virtualized container of the medical data system, wherein the management system is arranged with a user interface for managing the medical data system; a configuration unit for configuring the virtualized container in the N sub-nodes to form a distributed data system; and a replication unit for storing the medical data in a hospital information system in the distributed data system, to facilitate a user to operate the user interface to acquire the medical data required for conducting medical data analysis via the distributed data system.

Further, the virtualized container comprises a database for data storage and a data replication application for data replication, and the replication unit is further used for sending a data replication instruction to at least one of the N sub-nodes, such that the sub-node receiving the data replication instruction stores first medical data into the database via the data replication application, wherein the first medical data is part of the medical data in the hospital information system.

Further, the management system further comprises: an address acquisition unit for receiving the database address information of the hospital information system inputted by the user on the user interface; and an addition unit for carrying the database address information in the data replication instruction.

Further, the virtualized container comprises a data computation application for data computation and a data analysis application for data analysis, and the management system further comprises: an analysis instruction acquisition unit for receiving a data analysis instruction triggered by the user on the user interface, in which data analysis instruction is comprised the feature information of the medical data analysis for this time; an allocation unit for formulating M analysis tasks for accomplishing the medical data analysis for this time according to the data analysis instruction, wherein M is an integer greater than 0 and less than or equal to N; and sending the M analysis tasks to M sub-nodes of the N sub-nodes, such that the sub-nodes receiving the analysis tasks invoke the data computation application and the data analysis application to perform the received analysis tasks to obtain an analysis result; and a display unit for displaying the analysis result on the user interface.

Further, the configuration unit is further used for acquiring the address information of each of the N sub-nodes inputted by the user on the user interface; and configuring the virtualized container in a corresponding sub-node according to the address information of each sub-node.

### BRIEF DESCRIPTION OF THE DRAWINGS

FIG. 1 is an architecture diagram of a medical data system provided by an embodiment of the invention;

FIG. 2 is a first flow diagram of a medical data management method provided by an embodiment of the invention;

FIG. 3 is a second flow diagram of a medical data management method provided by an embodiment of the invention;

FIG. 4 is a first structure diagram of a management mainframe provided by an embodiment of the invention;

FIG. 5 is a second structure diagram of a management mainframe provided by an embodiment of the invention;

FIG. 6 is a third structure diagram of a management mainframe provided by an embodiment of the invention;

FIG. 7 is a fourth structure diagram of a management mainframe provided by an embodiment of the invention; and

FIG. 8 is a structure diagram of a computer device provided by an embodiment of the invention.

### DETAILED DESCRIPTION OF THE INVENTION

In the following the technical solutions in embodiments of the invention will be described clearly and completely in connection with the drawings in the embodiments of the invention. Obviously, the described embodiments are just a part of the embodiments of the invention, and not all the embodiments.

In addition, the term “first” or “second” is only used for the purpose of description, and cannot be understood as indicating or implying relative importance or implicitly specifying the number of an indicated technical feature. Thus, a feature defined by “first” or “second” may explicitly or implicitly comprise one or more said feature. In the description of the disclosure, the meaning of “a plurality of” is two or more than two.

An embodiment of the invention provides a medical data management method applicable in a medical data system 100 as shown in FIG. 1, which medical data system 100 comprises a management mainframe 11 and N sub-nodes 12 all connected with the management mainframe 11, wherein N is an integer greater than 0.

For example, the medical data system 100 may be deployed throughout a hospital or in a department of the hospital. One computer of the hospital or the department is arranged as the management mainframe 11, and other computers are the sub-nodes 12. As such, by a virtualization technique, it may be possible to take the whole medical data system 100 as a distributed big data development platform, store corresponding medical data, conduct resource integration, and thereby help a user (e.g., a doctor, a medical teacher, etc.) collect and enter relevant medical data via the platform when conducting medical data analysis, reduce the complexity of the medical data analysis, and improve the accuracy of the medical data analysis by conducting the medical data analysis by means of more comprehensive medical data.

Further, utilizing the existing big data analysis software, it may also be possible to integrate the data analysis function into the platform, for example, embed a machine learning tool or application into an individual sub-node for conducting a data mining task. As such, based on a great amount of medical data stored in the platform, and in combination with feature information selected by the user, such as an analysis object, an analysis problem and an analysis algorithm, etc., the medical data system 100 may invoke the machine learning tool or application to conduct the medical data analysis, which may realize a relatively friendly medical data analysis process for a user with limited computer expertise.

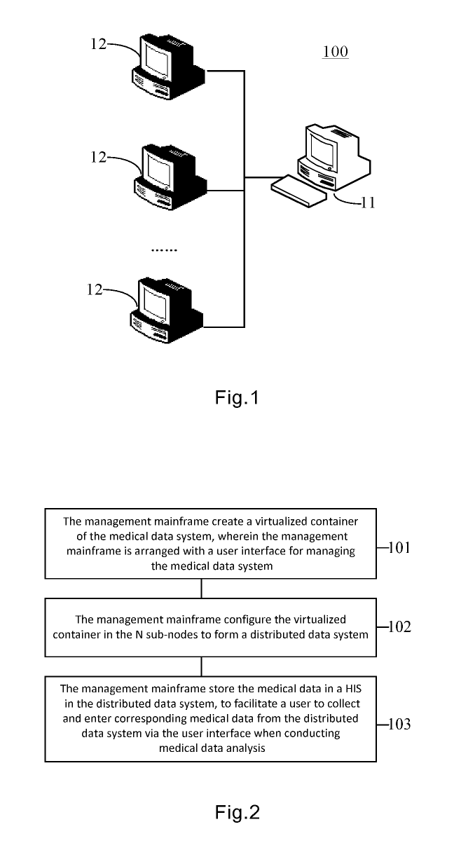

In particular, as shown in FIG. 2, an embodiment of the invention provides a medical data management method comprising the following steps.

At step 101, the management mainframe creates a virtualized container of the medical data system, wherein the management mainframe is arranged with a user interface for managing the medical data system.

Therein, the management mainframe is the core of the whole medical data system 100, and may be used for adding a sub-node and controlling a sub-node to implement a function of data storage, data analysis, and so on.

In particular, by installing main console software in the management mainframe, it may be possible to generate a user interface (UI) for managing the medical data system, and create a virtualized container of the medical data system. Any operation of the whole medical data system 100 may be carried out via a functional key on the user interface, and the created virtualized container is prepackaged with corresponding functional modules or applications, for example, an underlying data system (e.g., a file system, on which a database system generally relies as its most underlying storage) which may be used for storing data, a database (e.g., a structuralized database) for storing data, a computation module for computation, a data replication application for data replication, a data analysis application (e.g., the Oracle) for data analysis, and so on, which functional modules or applications will be automatically installed and configured in the process of creating the virtualized container.

Therein, the reason why a virtualized container is created in the management mainframe is that the isolation performance and the migration performance of the virtualized container are relatively good. Taking that a Docker container acts as a virtualized container as an example, by utilizing the Docker technique, a developer may be let to pack applications that need to be configured into a transplantable Docker container and then release them onto any Linux machine. Since a Docker container does not rely on any language, framework and system, the Docker container may be run on any computer and may be isolated from other data in a host (i.e., a computer where it is installed), that is, it will not affect the original functions of the host. Therefore, a virtualized container is created in the management mainframe, which will not affect the normal functions of the management mainframe, but may also conveniently configure a Docker container on other sub-node.

It needs to be noted that those skilled in the art may select the functional modules or applications prepackaged in the virtualized container according to the actual situation, which will not be defined by the embodiments of the invention in any way.

At step 102, the management mainframe configures the virtualized container in the N sub-nodes to form a distributed data system.

In particular, the user may input in the created user interface the address information of a sub-node that needs to be added, for example, the IP of the sub-node or the identification of the sub-node, etc., and then the management mainframe configures the virtualized container in a corresponding sub-node according to the inputted address information of each sub-node, eventually forming a distributed data system. Since the distributed data system may divide a computation instruction or an analysis instruction issued by the user each time into many small parts, which are allocated by the management mainframe to multiple sub-nodes for processing, this saves the overall data processing time and improves the data processing efficiency.

In addition, when the management mainframe configures the virtualized container in an individual sub-node, it may not just simply replicate the virtualized container in the management mainframe into the sub-node. Since some parameters for running the virtualized container may need to match those of the host (i.e., a corresponding sub-node), and yet parameters such as the memory sizes, the CPU main frequencies, etc. of different sub-nodes may be different, it is necessary to modify the parameters of the virtualized container accordingly when configuring the virtualized container in an individual sub-node, such that the virtualized container may be run normally in the individual sub-node.

Further, after the distributed data system is formed, it may further be possible to add or delete a sub-node in the distributed data system, so as to meet the storage or computing needs of the distributed data system.

For example, when it is required to add a sub-node, the user may trigger a corresponding functional key in the user interface, and at this point, the management mainframe generates and acquires an add-sub-node instruction, which carries the address information of a newly added sub-node. Then, similarly to the above configuration process, the management mainframe may configure the virtualized container in the newly added sub-node according to the address information of the newly added sub-node.

Or alternatively, when it is required to delete a sub-node, the user may trigger a corresponding functional key in the user interface, and at this point, the management mainframe generates and acquires a delete-sub-node instruction, which carries the address information of a to-be-deleted sub-node. Then, the management mainframe may delete the virtualized container configured in the to-be-deleted sub-node according to the address information of the to-be-deleted sub-node.

At step 103, the management mainframe stores the medical data in a HIS in the distributed data system, to facilitate a user to operate the user interface to acquire the medical data required for conducting medical data analysis via the distributed data system.

In particular, the virtualized container created at step 101 may comprise a database for data storage and a data replication application for data replication. For example, the database is a structuralized database, and the data replication application is used for replicating the medical data in the HIS into the structuralized database.

At this point, after the distributed data system is formed, based on a distributed storage technique, the management mainframe may send a data replication instruction to at least one of the N sub-nodes, such that the sub-node receiving the data replication instruction invokes the data replication application and stores first medical data into the database, wherein the first medical data is part of the medical data in the HIS. As such, each sub-node is used for storing a part of the medical data in the HIS, and eventually all of the medical data in the HIS is replicated in the whole distributed data system.

Thus, when the user conduct the medical data analysis subsequently, he may trigger a data analysis instruction on the user interface directly, and in turn, the management mainframe collects and enters corresponding medical data from the distributed data system according to the data analysis instruction.

For example, when the user needs to analyze the relationship between lung cancer and smoking, he may trigger a data analysis instruction on the user interface, for example, input on the user interface feature information that the disease is lung cancer, there is a history of smoking, and the sex is male, and the like, and then the management mainframe generates the data analysis instruction according to the feature information, and utilizes a distributed computing technique to instruct a corresponding sub-node to collect medical data that meets the feature information in the database, to facilitate the user to conduct the medical data analysis according to the search result. Moreover, when the distributed data system has the data analysis function, the management mainframe may further directly enter the collected medical data into a corresponding data analysis module, which simplifies the process of data collection and entry by the user at the time of the medical data analysis, and reduces the complexity of the medical data analysis.

Further, the virtualized container created at step 101 may further comprise a data computation application for data computation and a data analysis application for data analysis. For example, the data computation application may be any data computation strategy based on the distributed data system, and the data analysis application may be any big data analysis application, etc., which will not be limited by the embodiments of the invention in any way.

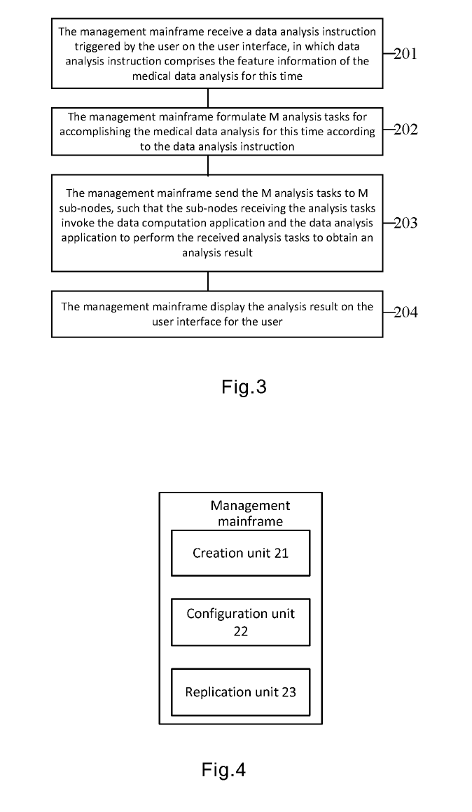

In particular, based on the above steps 101-103, when the virtualized container comprises a data computation application for data computation and a data analysis application for data analysis, after executing the step 103, the medical data management method may further comprise the following steps 201-204, as shown in FIG. 3.

At step 201, the management mainframe receives a data analysis instruction triggered by the user on the user interface, in which data analysis instruction is comprised the feature information of the medical data analysis for this time.

Since the analysis problem or the analysis object is different each time the medical data analysis is conducted, for medical data analysis for one time (i.e., the medical data analysis for this time), the user needs to input in the user interface the feature information of the medical data analysis for this time. For example, when the user needs to analyze the relationship between lung cancer and smoking, he needs to input on the user interface constraint conditions that the disease is lung cancer, there is a history of smoking, and the sex is male, and the like, which constraint conditions are right the feature information of the medical data analysis for this time, and in turn, the management mainframe generates the data analysis instruction according to the feature information.

At step 202, the management mainframe formulates M analysis tasks for accomplishing the medical data analysis for this time according to the data analysis instruction, wherein M is an integer greater than 0 and less than or equal to N.

In particular, according to the data analysis instruction carrying the feature information, the management mainframe needs to formulate M analysis tasks for accomplishing the medical data analysis for this time according to a certain data analysis strategy.

Therein, the M analysis tasks may be mutually independent logically. For example, it is formulated that the analysis task 1 is to find the age distribution of patients suffering from lung cancer, the analysis task 2 is to find the sex ratio of patients suffering from lung cancer, the analysis task 3 is to find the number of times of smoking of patients suffering from lung cancer, and so on, and these analysis tasks are mutually independent.

Or alternatively, the M analysis tasks may be logically progressive. For example, it is formulated that the analysis task 1 is to find all patients suffering from lung cancer, the analysis task 2 is to find whether the patients with lung cancer smoke on the basis of the result of the task 1, the analysis task 3 is to find the degrees of smoking of the patients with lung cancer that have a history of smoking, and these analysis tasks are interrelated.

It needs to be noted that those skilled in the art may formulate the data analysis strategy according to the actual situation or using different data analysis software, which will not be limited by the embodiments of the invention in any way.

At step 203, the management mainframe sends the M analysis tasks to M sub-nodes of the N sub-nodes, such that the sub-nodes receiving the analysis tasks invoke the data computation application and the data analysis application to perform the received analysis tasks to obtain an analysis result.

In particular, the management mainframe sends the M analysis tasks formulated at step 202 to M sub-nodes in the distributed data system, respectively. As such, each sub-node receiving an analysis task may acquire corresponding medical data in the distributed data system according to its own analysis task, and then invoke the data computation application and the data analysis application to perform the received analysis task.

At this point, if the M analysis tasks are mutually independent, M analysis results obtained by the M sub-nodes are not final analysis results, and the management mainframe may use the data computation application and the data analysis application to determine a final analysis result according to the M analysis results, or specify a corresponding sub-node to determine the final analysis result.

If the M analysis tasks are progressive, then the analysis result outputted by a sub-node responsible for the last one of the M analysis tasks is just the final analysis result.

At step 204, the management mainframe displays the analysis result on the user interface.

Here, what is displayed by the management mainframe on the user interface may be the final analysis result, or also may be the analysis result of each analysis task, so as to help the user conduct the medical data analysis.

So far, the embodiments of the invention provide a medical data management method applied in a medical data system, which system comprises a management mainframe and N sub-nodes all connected with the management mainframe, wherein the management mainframe creates a user interface for managing the medical data system and a virtualized container; and then, configures the virtualized container in the N sub-nodes to form a distributed data system; and subsequently, the management mainframe stores the medical data in a HIS in the distributed data system, in order that a user may directly operate the user interface to acquire the medical data required for conducting medical data analysis from the distributed data system when conducting the medical data analysis, it is unnecessary for the user to manually collect corresponding medical data from the HIS, it is also unnecessary for the user to have higher computer skills, the medical data analysis process is caused to be more friendly, it may not only be possible to simplify the data acquisition process at the time of medical data analysis and reduce the complexity of the medical data analysis, but also the acquired medical data is more comprehensive and the accuracy of the medical data analysis may be improved.

FIG. 4 is a structure diagram of a management mainframe provided by an embodiment of the invention. The management mainframe provided by the embodiment of the invention may be used for carrying out the method implemented by individual embodiments of the invention as shown in FIGS. 1-3. For the convenience of description, only the part relevant to the embodiment of the invention is shown, and for the specific technical details not disclosed, reference is made to the individual embodiments of the invention as shown in FIGS. 1-3.

In particular, as shown in FIG. 4, the management mainframe comprises: a creation unit 21 for creating a virtualized container of the medical data system, wherein the management mainframe is arranged with a user interface for managing the medical data system; a configuration unit 22 for configuring the virtualized container in the N sub-nodes to form a distributed data system; and a replication unit 23 for storing the medical data in a HIS in the distributed data system, to facilitate a user to operate the user interface to acquire the medical data required for conducting medical data analysis via the distributed data system.

Further, the virtualized container comprises a database for data storage and a data replication application for data replication; and the replication unit 23 is specifically used for sending a data replication instruction to at least one of the N sub-nodes, such that the sub-node receiving the data replication instruction stores first medical data into the database via the data replication application, wherein the first medical data is part of the medical data in the HIS.

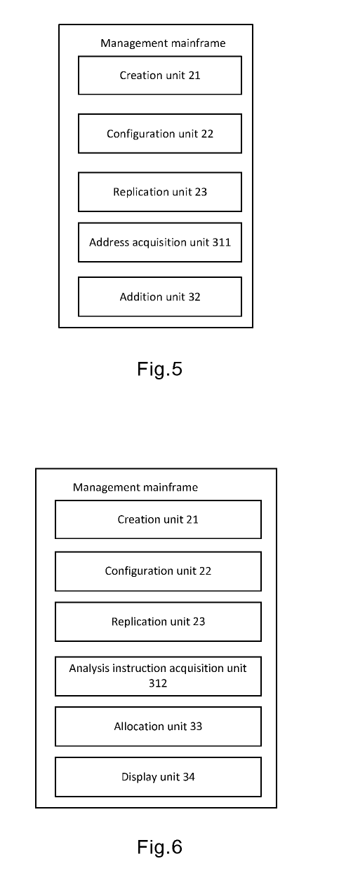

Further, as shown in FIG. 5, besides the individual units comprised in FIG. 4, the management mainframe comprises: an address acquisition unit 311 for receiving the database address information of the HIS inputted by the user on the user interface; and an addition unit 32 for carrying the database address information in the data replication instruction.

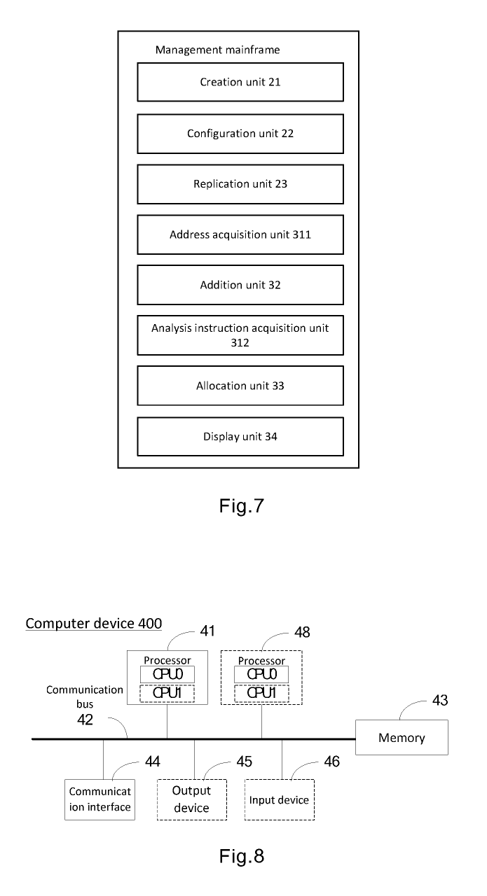

Further, the virtualized container comprises a data computation application for data computation and a data analysis application for data analysis. As shown in FIG. 6 or FIG. 7, besides the individual units comprised in FIG. 4 or FIG. 5, the management mainframe comprises: an analysis instruction acquisition unit 312 for receiving a data analysis instruction triggered by the user on the user interface, in which data analysis instruction is comprised the feature information of the medical data analysis for this time; an allocation unit 33 for formulating M analysis tasks for accomplishing the medical data analysis for this time according to the data analysis instruction, wherein M is an integer greater than 0 and less than or equal to N; and sending the M analysis tasks to M sub-nodes of the N sub-nodes, such that the sub-nodes receiving the analysis tasks invoke the data computation application and the data analysis application to perform the received analysis tasks to obtain an analysis result; and a display unit 34 for displaying the analysis result on the user interface for the user.

Further, the configuration unit 22 is further used for acquiring the address information of each of the N sub-nodes inputted by the user on the user interface; and configuring the virtualized container in a corresponding sub-node according to the address information of each sub-node.

Further, the configuration unit 22 is further used for acquiring an add-sub-node instruction, which carries the address information of a newly added sub-node, and configuring the virtualized container in the newly added sub-node according to the address information of the newly added sub-node; and is further used for acquiring a delete-sub-node instruction, which carries the address information of a to-be-deleted sub-node, and deleting the virtualized container configured in the to-be-deleted sub-node according to the address information of the to-be-deleted sub-node.

Exemplarily, the management mainframe as shown in FIGS. 4-6 may be implemented in the form of the computer device (or system) in FIG. 8.

What is shown in FIG. 8 is a schematic diagram of a computer device provided by an embodiment of the invention. The computer device 400 comprises at least one processor 41, a communication bus 42, a memory 43 and at least one communication interface 44.

For example, the specific functions of the creation unit 21, the configuration unit 22, the replication unit 23, the address acquisition unit 311, the analysis instruction acquisition unit 312, the addition unit 32, the allocation unit 33, and the display unit 34 described above may be realized by the processor 41 in the computer device invoking computer instructions in the memory 43.

In particular, the processor 41 may be a general-purpose central processing unit (CPU), a microprocessor, an application-specific integrated circuit (ASIC), or one or more integrated circuit for controlling execution of programs corresponding to the embodiments of the disclosure.

The communication bus 42 may comprise at least one pathway for passing information between the above components. The communication interface 44 uses any apparatus of the type of transceiver for communicating with other device or communication network, for example, the Ethernet, the wireless access network (RAN), the wireless local area network (WLAN), etc.

The memory 43 may be a read-only memory (ROM) or other type of static storage device which may store static information and instructions, a random access memory

(RAM) or other type of dynamic storage device which may store information and instructions, or also an electrically erasable programmable read-only memory (EEPROM), a compact disc read-only memory (CD-ROM) or other optical disk storage, optical disc storage (comprising compressed disc, laser disc, optical disc, digital versatile disc, blu-ray disc, etc.), a magnetic disk storage medium or other magnetic storage device, or any other medium that can be used for carrying or storing a desired program code in the form of instructions or a data structure and can be accessed by a computer. However, it is not limited thereto. The memory 43 may be stand-alone, and connected with the processor via the bus. The memory 43 may also be integrated with the processor.

Therein, the memory 43 is used for storing a corresponding application code implementing an embodiment of the invention, and the implementation is controlled by the processor 41. The processor 41 is used for executing the application code stored in the memory 43.

In a specific implementation, as an embodiment, the processor 41 may comprise one or more CPU, e.g., the CPU0 and the CPU1 in FIG. 8.

In a specific implementation, as an embodiment, the computer device may comprise a plurality of processors, e.g., the processor 41 and the processor 48 in FIG. 7. Each of the processors may be a single-core processor, or also a multi-core processor. The processor here may refer to one or more device, circuit and/or processing core for processing data (e.g., a computer program instruction).

In a specific implementation, as an embodiment, the computer device may further comprise an output device 45 and an input device 46. The output device 45 communicates with the processor 41 and may display information in multiple ways. For example, the output device 45 may be a liquid crystal display (LCD), a light emitting diode (LED) display device, a cathode ray tube (CRT) display device, or a projector, etc. The input device 46 communicates with the processor 41 and may receive an input from the user in multiple ways. For example, the input device 46 may be a mouse, a keyboard, a touch screen device or a sensor device, etc.

The above described computer device may be a general-purpose computer device or a dedicated computer device. In a specific implementation, the computer device may be a desktop computer, a portable computer, a network server, a personal digital assistant (PDA), a mobile phone, a tablet computer, a wireless terminal device, a communication device, an embedded device or a device with a structure similar to FIG. 8. The embodiments of the invention do not define the type of the computer device.

It needs to be noted that in an example of the invention, any of the functional nodes in the medical data system 100 as described above, for example, the management mainframe 11, the sub-node 12, may be implemented by one entity device, or also may be implemented jointly by multiple entity devices, and the individual functional nodes in the medical data system 100 may be implemented by different entity devices, respectively, or also may be implemented by one and the same entity device. It will be appreciated that any of the functional nodes in the medical data system 100 may be a logical functional module in an entity device, or also may be a logical functional module constituted by multiple entity devices.

Hitherto, the embodiments of the invention provide a management mainframe applied in a medical data system, which system comprises the management mainframe and N sub-nodes all connected with the management mainframe, wherein the management mainframe creates a virtualized container of the medical data system, and the management mainframe is arranged with a user interface for managing the medical data system; and then, it configures the virtualized container in the N sub-nodes to form a distributed data system; and subsequently, the management mainframe stores the medical data in a HIS in the distributed data system, in order that a user may directly operate the user interface to acquire the medical data required for conducting medical data analysis from the distributed data system when conducting the medical data analysis, it is unnecessary for the user to manually collect corresponding medical data from the HIS, it is also unnecessary for the user to have higher computer skills, the medical data analysis process is caused to be more friendly, it may not only be possible to simplify the data acquisition process at the time of medical data analysis and reduce the complexity of the medical data analysis, but also the acquired medical data is more comprehensive and the accuracy of the medical data analysis may be improved.

In the description of the specification, specific features, structures, materials or characteristics may be combined in an appropriate way in any one or more of the embodiments or examples.

What are described above are just specific embodiments of the invention, however, the protection scope of the invention is not limited thereto, and variations or alternatives easily occurring to any artisan familiar with the technical field within the technical scope disclosed by the invention should be encompassed within the protection scope of the invention. Therefore, the protection scope of the invention should be subject to the protection scope of the claims.

1. A management mainframe applied in a medical data system, which medical data system comprises the management mainframe and N sub-nodes all connected with the management mainframe, wherein N is an integer greater than 0, the management mainframe comprising:
a storage for storing data and an instruction, and
a processor configured to, when the instruction is executed in the processor, implement the following steps of:
creating a virtualized container of the medical data system, wherein the management mainframe is arranged with a user interface for managing the medical data system;
configuring the virtualized container in the N sub-nodes to form a distributed data system; and
storing the medical data in a hospital information system in the distributed data system, to facilitate a user to operate the user interface to acquire the medical data required for conducting medical data analysis via the distributed data system.

2. The management mainframe as claimed in claim 1, wherein the virtualized container comprises a database for data storage and a data replication application for data replication, and
the processor is further configured to send a data replication instruction to at least one of the N sub-nodes, such that the sub-node receiving the data replication instruction stores first medical data into the database via the data replication application, wherein the first medical data is part of the medical data in the hospital information system.

3. The management mainframe as claimed in claim 2, wherein the processor is further configured to:
receive the database address information of the hospital information system inputted by the user on the user interface; and
carry the database address information in the data replication instruction.

4. The management mainframe as claimed in claim 1, wherein the virtualized container comprises a data computation application for data computation and a data analysis application for data analysis, and the processor is further configured to:
receive a data analysis instruction triggered by the user on the user interface, in which data analysis instruction is comprised the feature information of the medical data analysis for this time;
formulate M analysis tasks for accomplishing the medical data analysis for this time according to the data analysis instruction, wherein M is an integer greater than 0 and less than or equal to N;
send the M analysis tasks to M sub-nodes of the N sub-nodes, such that the sub-nodes receiving the analysis tasks invoke the data computation application and the data analysis application to perform the received analysis tasks to obtain an analysis result; and
display the analysis result on the user interface.

5. The management mainframe as claimed in claim 1, wherein the processor is further configured to:
acquire the address information of each of the N sub-nodes inputted by the user on the user interface; and
configure the virtualized container in a corresponding sub-node according to the address information of each sub-node.

6. The management mainframe as claimed in claim 1, wherein the processor is further configured to, after configuring the virtualized container in the N sub-nodes to form a distributed data system,
acquire an add-sub-node instruction, which carries the address information of a newly added sub-node; and
configure the virtualized container in the newly added sub-node according to the address information of the newly added sub-node.

7. The management mainframe as claimed in claim 1, wherein the processor is further configured to, after configuring the virtualized container in the N sub-nodes to form a distributed data system,
acquire a delete-sub-node instruction, which carries the address information of a to-be-deleted sub-node; and
delete the virtualized container configured in the to-be-deleted sub-node according to the address information of the to-be-deleted sub-node.

8. (canceled)

9. A medical data system comprising a management mainframe as claimed in claim 1 and N sub-nodes all connected with the management mainframe, wherein N is an integer greater than 0.

10. A medical data management method, wherein the method is applied in a medical data system, which medical data system comprises a management mainframe and N sub-nodes all connected with the management mainframe, wherein N is an integer greater than 0, and the method comprises:
the management mainframe creating a virtualized container of the medical data system, wherein the management mainframe is arranged with a user interface for managing the medical data system;
the management mainframe configuring the virtualized container in the N sub-nodes to form a distributed data system; and
the management mainframe storing the medical data in a hospital information system in the distributed data system, to facilitate a user to acquire the medical data required for conducting medical data analysis via the distributed data system when operating the user interface.

11. The method as claimed in claim 10, wherein the virtualized container comprises a database for data storage and a data replication application for data replication,
wherein the management mainframe storing the medical data in the hospital information system in the distributed data system comprises:
the management mainframe sending a data replication instruction to at least one of the N sub-nodes, such that the sub-node receiving the data replication instruction stores first medical data into the database via the data replication application, wherein the first medical data is part of the medical data in the hospital information system.

12. The method as claimed in claim 11, wherein before the management mainframe sending a data replication instruction to at least one of the N sub-nodes, there is further comprised:
the management mainframe receiving the database address information of the hospital information system inputted by the user on the user interface; and
the management mainframe carrying the database address information in the data replication instruction.

13. The method as claimed in claim 10, wherein the virtualized container comprises a data computation application for data computation and a data analysis application for data analysis,
wherein after the management mainframe storing the medical data in the hospital information system in the distributed data system, there is further comprised:
the management mainframe receiving a data analysis instruction triggered by the user on the user interface, in which data analysis instruction is comprised the feature information of the medical data analysis for this time;
the management mainframe formulating M analysis tasks for accomplishing the medical data analysis for this time according to the data analysis instruction, wherein M is an integer greater than 0 and less than or equal to N;
the management mainframe sending the M analysis tasks to M sub-nodes of the N sub-nodes, such that the sub-nodes receiving the analysis tasks invoke the data computation application and the data analysis application to perform the received analysis tasks to obtain an analysis result; and
the management mainframe displaying the analysis result on the user interface.

14. The method as claimed in claim 10, wherein the management mainframe configuring the virtualized container in the N sub-nodes comprises:
the management mainframe acquiring the address information of each of the N sub-nodes inputted by the user on the user interface; and
the management mainframe configuring the virtualized container in a corresponding sub-node according to the address information of each sub-node.

15. The method as claimed in claim 10, wherein after the management mainframe configuring the virtualized container in the N sub-nodes to form a distributed data system, there is further comprised:
the management mainframe acquiring an add-sub-node instruction, which carries the address information of a newly added sub-node; and
the management mainframe configuring the virtualized container in the newly added sub-node according to the address information of the newly added sub-node.

16. The method as claimed in claim 10, wherein after the management mainframe configuring the virtualized container in the N sub-nodes to form a distributed data system, there is further comprised:
the management mainframe acquiring a delete-sub-node instruction, which carries the address information of a to-be-deleted sub-node; and
the management mainframe deleting the virtualized container configured in the to-be-deleted sub-node according to the address information of the to-be-deleted sub-node.

17. (canceled)

18. A management system applied in a medical data system, which medical data system comprises the management system and N sub-nodes all connected with the management system, wherein N is an integer greater than 0, the management system comprising:
a creation unit for creating a virtualized container of the medical data system, wherein the management system is arranged with a user interface for managing the medical data system;
a configuration unit for configuring the virtualized container in the N sub-nodes to form a distributed data system; and
a replication unit for storing the medical data in a hospital information system in the distributed data system, to facilitate a user to operate the user interface to acquire the medical data required for conducting medical data analysis via the distributed data system.

19. The management system as claimed in claim 18, wherein the virtualized container comprises a database for data storage and a data replication application for data replication, and
the replication unit is further used for sending a data replication instruction to at least one of the N sub-nodes, such that the sub-node receiving the data replication instruction stores first medical data into the database via the data replication application, wherein the first medical data is part of the medical data in the hospital information system.

20. The management system as claimed in claim 19, wherein the management system further comprises:
an address acquisition unit for receiving the database address information of the hospital information system inputted by the user on the user interface; and
an addition unit for carrying the database address information in the data replication instruction.

21. The management system as claimed in claim 18, wherein the virtualized container comprises a data computation application for data computation and a data analysis application for data analysis, and the management system further comprises:
an analysis instruction acquisition unit for receiving a data analysis instruction triggered by the user on the user interface, in which data analysis instruction is comprised the feature information of the medical data analysis for this time;
an allocation unit for formulating M analysis tasks for accomplishing the medical data analysis for this time according to the data analysis instruction, wherein M is an integer greater than 0 and less than or equal to N; and sending the M analysis tasks to M sub-nodes of the N sub-nodes, such that the sub-nodes receiving the analysis tasks invoke the data computation application and the data analysis application to perform the received analysis tasks to obtain an analysis result; and
a display unit for displaying the analysis result on the user interface.

22. The management system as claimed in claim 18, wherein
the configuration unit is further used for acquiring the address information of each of the N sub-nodes inputted by the user on the user interface; and configuring the virtualized container in a corresponding sub-node according to the address information of each sub-node.
